(19) United States
(12) Patent Application Publication
(10) Pub. No.: US 2023/0338348 A1
(43) Pub. Date: Oct. 26, 2023

(54) RENIN-ANGIOTENSIN SYSTEM (RAS) MODULATORS FOR TREATMENT OF VIRAL INFECTIONS, PHARMACEUTICAL COMPOSITIONS INCLUDING THE SAME, AND METHODS OF TREATING USING THE SAME

(71) Applicant: Trocar Pharma Inc., Laurel, MD (US)

(72) Inventors: Milton L. BROWN, Brookeville, MD (US); Courtney W. HOUCHEN, Oklahoma City, OK (US); Yali KONG, Fairfax, VA (US)

(73) Assignee: Trocar Pharma Inc., Laurel, MD (US)

(21) Appl. No.: 17/996,678

(22) PCT Filed: Apr. 21, 2021
(86) PCT No.: PCT/US2021/028357

§ 371 Date: Oct. 20, 2022

Related U.S. Application Data
(60) Provisional application No. 63/012,981, filed on Apr. 21, 2020.

(51) Int. Cl. A61K 31/4418; A61K 38/08; A61K 47/55; A61K 31/445; A61P 39/06; A61P 31/14

(52) U.S. Cl. CPC A61K 31/4418; A61K 38/085; A61K 47/55; A61K 31/445; A61P 39/06; A61P 31/14

(57) ABSTRACT

A method of treating a viral infection may include administering a pharmaceutical composition including a renin-angiotensin system (RAS) modulator to a subject in need thereof to mitigate a cellular and organic impact of the viral infection. The mitigation may include inhibiting reactive oxygen species, inhibiting cytokine release, upregulating angiotensin-converting enzyme 2 (ACE2), and/or downregulating angiotensin II receptor type 1 (AT1). The renin-angiotensin system modulator may include various combinations of an angiotensin receptor blocker (ARB), angiotensin (1-7), an HMG-CoA reductase inhibitor, an angiotensin-converting-enzyme (ACE) inhibitor, 3,3′-diindolylmethane (DIM), indole-3-carbinol (I3C), and/or pirfenidone (PFD). In addition, at least one of the angiotensin receptor blocker, the angiotensin (1-7), the HMG-CoA reductase inhibitor, the angiotensin-converting-enzyme inhibitor, 3,3′-diindolylmethane, indole-3-carbinol, or pirfenidone may be linked to an antioxidant.

### BACKGROUND

### Field

The present disclosure relates to renin-angiotensin system (RAS) modulators, pharmaceutical compositions including RAS modulators, and methods for the treatment of viral infections.

### Description of Related Art

Vaccines can prevent certain viral diseases, and antiviral drugs may interfere with the reproduction of viruses and/or strengthen the immune response to certain viral infections. However, there are still no effective antiviral drugs for many viral infections. As a result, for most viral infections, treatments can only help with the symptoms while waiting for the immune system to fight off the virus. The emergence of the severe acute respiratory syndrome coronavirus 2 (SARS-CoV-2) and ensuing COVID-19 pandemic has highlighted the havoc that viruses can cause when the requisite vaccines and antiviral drugs are not available.

### SUMMARY

At least one embodiment relates to a method of treating a viral infection. In an example embodiment, the method may include administering a pharmaceutical composition including a renin-angiotensin system (RAS) modulator to a subject in need thereof to mitigate a cellular and organic impact of the viral infection. The mitigation may include inhibiting reactive oxygen species, inhibiting cytokine release, upregulating angiotensin-converting enzyme 2 (ACE2), and/or downregulating angiotensin II receptor type 1 (AT1).

At least one embodiment relates to a pharmaceutical composition for treating a viral infection. In an example embodiment, the pharmaceutical composition may include a renin-angiotensin system (RAS) modulator and a pharmaceutically-acceptable carrier. The RAS modulator may include various combinations of an angiotensin receptor blocker (ARB), angiotensin (1-7), an HMG-CoA reductase inhibitor, an angiotensin-converting-enzyme (ACE) inhibitor, 3,3′-diindolylmethane (DIM), indole-3-carbinol (I3C), and/or pirfenidone (PFD). In addition, at least one of the angiotensin receptor blocker, the angiotensin (1-7), the HMG-CoA reductase inhibitor, the angiotensin-converting-enzyme inhibitor, DIM, I3C, or pirfenidone may be linked to an antioxidant.

For instance, disclosed herein is a method of using an angiotensin receptor blocker tethered to an antioxidant (e.g., Tempol) for the treatment of viral infections. As an example, YK-4-250 may be used as an effective agent to prevent or inhibit complications of viral infection. As another example, the combination of YK-4-250 and a HMG-CoA reductase inhibitor may be used as an effective agent to prevent or inhibit complications of viral infection. Also disclosed is a method of using an angiotensin receptor blocker and a HMG-CoA inhibitor for the treatment of viral infections. As an example, Telmisartan and Rosuvastatin may be used as an effective agent to prevent or inhibit complications of viral infection. Also disclosed is a method of using DIM tethered to an antioxidant (e.g., Tempol) for the treatment of viral infections. DIM (and its precursor, I3C) has been demonstrated to inhibit RAS signaling induced by VEGF and other growth factors, which interferes with its downstream biological effects necessary for angiogenesis.¹ Further disclosed is a method of using pirfenidone (PFD) tethered to an antioxidant (e.g., Tempol) for the treatment of viral infections. PFD exerts anti-fibrotic effects through blockade of TGF-β promoter activity and TGF-β protein secretion, inhibition of TGF-β-induced Smad2-phosphorylation, ECM stimulation and ROS generation, and regulation of RNA processing. TGF-β1 activates RAS and mitogen-activated protein (MAP) kinases, the phosphoinositide 3-kinase (PI3K)/Akt pathway, and Rho GTPases, and regulates cell growth, survival, migration, and cytoskeleton organization.²

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a schematic overview of a renin-angiotensin system (RAS) activation and COVID-19 infection.

FIG. 2 is a bar graph displaying the inhibition of O₂₋ generation by YK-4-250.

FIG. 3 illustrates the synthesis of YK-4-250. ¹ Chang X, Firestone G L, Bjeldanes L F. Inhibition of growth factor-induced Ras signaling in vascular endothelial cells and angiogenesis by 3,3′-diindolylinethane. Carcinogenesis. 2006 Mar;27(3):5411-50. doi: 10.1093/carcin/bgi230. Epub 2005 Sep. 30. PMID: 16199440.² Shi, K., Wang, F., Xia, J., Zuo, B., Wang, Z.. & Cao, X. (2019). Pirfenidorie inhibits epidural scar fibroblast proliferation and differentiation by regulating TGE-β1-induced Smad-dependent and -independent pathways. American journal of translational research, 11(3), 1.593-1604.

FIG. 4 illustrates the synthesis of an antioxidant-TGF-β inhibitor.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives thereof. Like numbers refer to like elements throughout the description of the figures.

It should be understood that when an element or layer is referred to as being “on,” “connected to,” “coupled to,” “attached to,” “adjacent to,” “covering,” etc. another element or layer, it may be directly on, connected to, coupled to, attached to, adjacent to, covering, etc. the other element or layer or intervening elements or layers may be present. In contrast, when an element is referred to as being “directly on,” “directly connected to,” “directly coupled to,” etc. another element or layer, there are no intervening elements or layers present. Like numbers refer to like elements throughout the specification. As used herein, the term “and/or” includes any and all combinations or sub-combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

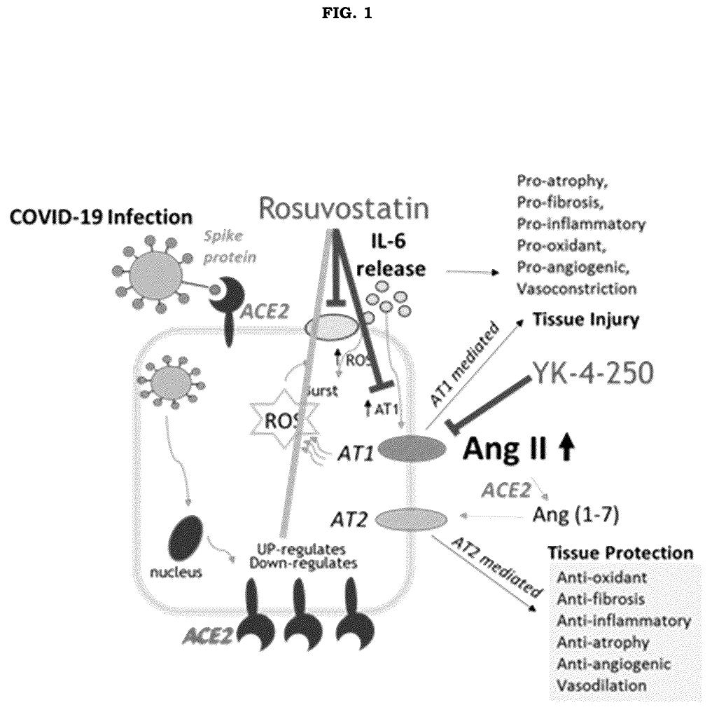

FIG. 1 is a schematic overview of a renin-angiotensin system (RAS) activation and COVID-19 infection. The angiotensin-converting enzyme 2 (ACE2) is the receptor for the COVID-19 infection and is expressed in the lung, the gastrointestinal (GI) tract, and the cardiovascular system. ACE2 is a key enzyme in the renin-angiotensin system (RAS) and inactivates angiotensin II (Ang II), a negative regulator of the system. ACE2 protects mice from severe acute lung injury induced by COVID-19 infection, acid aspiration, or sepsis. When Ang II binds to the AT1 receptor, it promotes reactive oxygen species (ROS) and inflammation resulting in lung tissue damage and impaired function (FIG. 1). Recombinant ACE2 can protect mice from acute lung injury mediated by severe acute respiratory syndrome coronavirus 2 (SARS-CoV-2) infection.¹ The critical protective function of the ACE2 receptor in acute lung injury and potential life-threatening damage to other organs, points to a possible therapy for COVID-19 infection.³ Increased RAS activation results in a surge of ROS escalating inflammation induced tissue damage. A therapeutic agent that inhibits viral activation of RAS and ROS following COVID-19 infection would have a major impact on morbidity and mortality.

The adverse effects of the virus may be mediated by boosting the anti-inflammatory, antioxidant, and anti-fibrotic response of the host on COVID-19 infection. For example, YK-4-250, a long acting combination of the angiotensin II receptor blocker (ARB) Telmisartan tethered to a highly potent antioxidant 4-hydroxy-TEMPO (as referred to as Tempol), will mitigate the adverse effects and improve survival in patients infected with COVID-19. YK-4-250 specifically binds to AT1 receptors on the lung, GI tract, and endothelium and potentially reverses the deleterious effects of viral mediated RAS and ROS activation. Furthermore, the reduction in ROS will temper the IL-6 release and quench the viral induced cytokine storm. Both Telmisartan and Tempol have been approved by the FDA for use in humans. However, Telmisartan (24 hour half-life) and Tempol (5 min half-life) cannot be co-administered as separate agents because of the large disparity in half-life. Thus, YK-4-250 (5.4 hour half-life) which administers both Telmisartan and Tempol as one agent is a desirable candidate for this novel indication. ³ Imai Y, Kuba K, Rao S, et al. Angiotensin-converting enzyme 2 protects from severe acute lung failure. Nature. 2005; 436(7047):112-116. doi:10.1038/nature03712.

Up-regulation of ACE2 is critical to fighting COVID-19 infection. ARBs up-regulate ACE2 and recent suggestions that ARBs might predispose patients to viral infection is without scientific support.⁴ Moreover, multiple studies have demonstrated that an increase in ACE2 is cytoprotective and mitigating of lung injuries from SARS-CoV-2 infection.^{1,5} When the COVID-19 virus enters the cell, it immediately downregulates the ACE2 protein expression which is critical for regulating RAS. Paradoxically, increasing ACE2 expression in the lung protects mice from SARS-CoV-2 spike protein-induced lung injury by attenuating the RAS. ACE2 also suppresses intestinal inflammation and is an antioxidant.⁶ Single cell-RNA sequencing data from colonocytes from normal patients and IBD patients demonstrated that ACE2 expression positively correlated with genes that regulate viral infection, innate and adaptive immunity, but was negatively associated with viral transcription, protein translation, and humoral immunity. Taken together, these data strongly suggest that increased ACE2 expression plays a complex role in viral infection and the immune response. The addition of a HMG-CoA reductase inhibitor like Rosuvastatin will up regulate ACE2 and work in synergy with the proposed therapy of AT1 inhibition.

COVID-19 injures the lung, GI tract, and cardiovascular system. Acute respiratory distress syndrome (ARDS), the most severe form of acute lung injury, is a devastating clinical syndrome with a high mortality rate (30-60%).¹ Predisposing factors ⁴ Lei Fang, George Karakiulakis, Michael Roth. Are patients with hypertension and diabetes mellitus at increased risk for COVID-19 infection? Lancet. Mar. 11, 2020DOI:https://doi.org/10.1016/S2213-2600(20)30116-8.⁵ Kuba, K., Imai, Y., Rao, S. et al. A crucial role of angiotensin converting enzyme 2 (ACE2) in SARS coronavirus-induced lung injury. Nat Med 11, 875-879 (2005). https://doi.org/10.1038/nm1267.⁶ Wang J, Zhao S, Liu M, et al. ACE2 expression by colonic epithelial cells is associated with viral infection, immunity and energy metabolism, 2020.

for ARDS are diverse and include sepsis, aspiration, pneumonias and infections with SARS-CoV-2. At present, there are no effective drugs for improving the clinical outcome of ARDS.

While local tissue-based Ang II exacerbates pulmonary hypertension, acute lung injury and lung fibrosis,⁷ experimental models have demonstrated that ACE2 mediates viral entry into the alveolar epithelial type II cells and its deficiency worsens lung injury by activating the RAS.³ The AT1 receptor located on the Dclk1+ tuft cells (unpublished data), when blocked, reduced SARS-coronavirus spike protein mediated lung injury⁶ and reduced pulmonary hypertension in experimental models.⁷ Spike protein engagement downregulates ACE2 expression and activates the RAS.⁶ Given that hypertension is common in severe SARS-CoV-2 pneumonia, it is highly likely that the RAS is activated in the lungs of patients with severe pneumonia.⁴ Down-regulation of cellular ACE2 expression following SARS-CoV-2 infection results in impaired function of ACE2 within the RAS. Since ACE2 is a prominent inhibitor of acute lung injury, a loss of ACE2 expression is thought to provoke the severe symptoms observed during infection with SARS-CoV-2. Evidence demonstrates that the binding of Ang II to the AT1 receptor promotes tissue damage by increasing inflammation, oxidative stress, fibrosis, angiogenesis and vasoconstriction.⁸ The ARB family specifically inhibits the binding of Ang II to the AT1 receptor and as such has the potential to reverse the constellation of adverse effects of COVID-19 infection.

In a small study of 204 patients diagnosed with COVID-19 in the Hubei province of China, researchers noted that nearly 49% of these patients presented with ⁷ Marshall R P. The pulmonary renin-angiotensin system. Curr Pharm Des 2003; 9:715-22.⁸ Benigni A, Cassis P, Remuzzi G. Angiotensin II revisited: new roles in inflammation, immunology and aging. EMBO Mol Med 2010; 2:247-57.

GI symptoms and these patients had adverse outcomes and reduced survival compared to those without GI symptoms.² ACE2 is the receptor for viral entry of the SARS-CoV-2 into the target cells. ACE2 interacts with the viral spike protein (FIG. 1) and mediates SARS-CoV-2 infection of the alveolar epithelial type II cells in the lung³ and colonic epithelial cells in the GI tract.³

In a study of 187 patients with COVID-19, 27.8% of patients had myocardial injury, which resulted in cardiac dysfunction and arrhythmias. Myocardial injury has been associated with fatal outcome of COVID-19 infection. Inflammation may be a potential mechanism for myocardial injury.⁹ The aggressive treatment to limit inflammation and ROS should be considered for patients at high risk of myocardial injury. A drug like YK-4-250 which quenches ROS would be a desirable candidate for these high risk patients.

Disclosed herein is a novel therapy that will block Ang II, up-regulate ACE2, and provide a relatively long acting antioxidant to infected and supportive cells that express Ang II AT1. The tethering of an antioxidant Tempol to the long acting Ang II AT1 blocker Telmisartan provides a new¹⁰ agent (YK-4-250) with antiviral, anti-inflammatory, and powerful antioxidant activity. The most intriguing aspect of YK-4-250 which makes it superior to traditional ARBs is the addition of the antiviral,¹¹ long ⁹ Guo T, Fan Y, Chen M, et al. Cardiovascular Implications of Fatal Outcomes of Patients With Coronavirus Disease 2019 (COVID-19). JAMA Cardiol. Mar. 27, 2020. doi: 10.1001/jamacardio.2020.1017.¹⁰ Brown M L, Kong, Y., Wilcox, C. S. Treatment for oxidative stress and/or hypertension. In: Patent US, ed. USPTO. USA: Georgetown University (Washington, DC), 2016.¹¹ David Olagnier, et al Cellular Oxidative Stress Response Controls the Antiviral and Apoptotic Programs in Dengue Virus-Infected Dendritic Cells. PLoS Pathog. 2014 Dec.; 10(12): e1004566.

acting, and highly potent antioxidant. Tempol has been shown to dramatically reduce cardiac oxidative damage and improve left ventricular dysfunction.¹²

Disclosed herein is a mitigator of the COVID-19 infection that has the potential to specifically attenuate the respiratory, GI tract, and cardiovascular manifestations of the infection. Relevant activities include scaling up production of YK-4-250, performing appropriate pre-clinical in vitro and in vivo studies on COVID-19, and completing a Phase 1 clinical trial.

Disclosed herein is non-clinical data as to YK-4-250 being a potent and selective inhibitor of in vitro Ang II AT1. YK-4-250 demonstrates selectivity and potency for the Ang II AT1 (1 nM inhibited about 47%) as compared to the AT2 subtype (10 nM resulted in about 5% inhibition) receptor inhibition (Table 1).

TABLE 1
Effects of YK-4-250 on angiotensin II receptors

| | % Inhibition | |
|---|---|---|
| | AT2^{a} | AT1^{b} |
| Telmisartan | 6.37% | 52.68% |
| YK-4-250 | 5.18% | 46.65% |

^{a}AT2, angiotensin II assay displays percent inhibition (N = 2) at 1.0 × 10⁻⁹M drug.
^{b}Human AT1 and percent inhibition (N = 2) at 1.0 × 10⁻⁸M drug

The potency and selectivity of YK-4-250 was comparable to clinically used Telmisartan. This data supports that the Ang II AT1 binding site tolerates the addition of the Tempol moiety. ¹² Aziz Guellich, Thibaud Damy, Marc Conti, Victor Claes, Jane-Lise Samuel, Thierry Pineau, Yves Lecarpentier, Catherine Coirault. Tempol Prevents Cardiac Oxidative Damage and Left Ventricular Dysfunction in the PPAR-α KO Mouse. Am J Physiol Heart Circ Physiol, 304 (11), H1505-12 2013.

The pharmacokinetic profile for YK-4-250 is disclosed herein. YK-4-250 was characterized in SD rats and provided in Table 2, YK-4-250 is an orally stable Tempol and has a t_{1/2} of 5.4 hours. As compared to the half-life of Tempol (less than 5 min),¹³ YK-4-250 is the first long-acting orally available Tempol derivative reported to date. ¹³ Kuppusamy P, Wang P, Shankar R A, et al. In vivo topical EPR spectroscopy and imaging of nitroxide free radicals and polynitroxyl-albumin. Magn Reson Med 1998; 40:806-11.

TABLE 2
Pharmacokinetic parameters for YK-4-250

| parameter | value | |
|---|---|---|
| Cmax | 0.45 | ug/ml |
| Tmax | 1.50 | h |
| AuClast | 2.82 | h*(ug/ml) |
| T_{1/2} | 5.40 | h |
| MRT | 8.67 | h |

Disclosed herein is a study regarding the maximal tolerated dose (MTD) of YK-4-250 in rodents. The single-dose acute toxicity of YK-4-250 was measured in male and female SD rats according to the acute oral toxicity (AOT) up and down procedure.²³ SD rats were purchased from the National Cancer Institute (NCI). YK-4-250 stock solution was prepared in water and the concentration was 200 mg/mL. YK-4-250 (100, 200, 300, 400, and 600 mg/kg) was administered orally, and uninterrupted observations were maintained for the first 4 h. The acute toxicity was observed daily for 14 days. Animals were sacrificed and all pathological findings were recorded. Single dose YK-4-250 MTD in SD rats was >550 mg/kg.

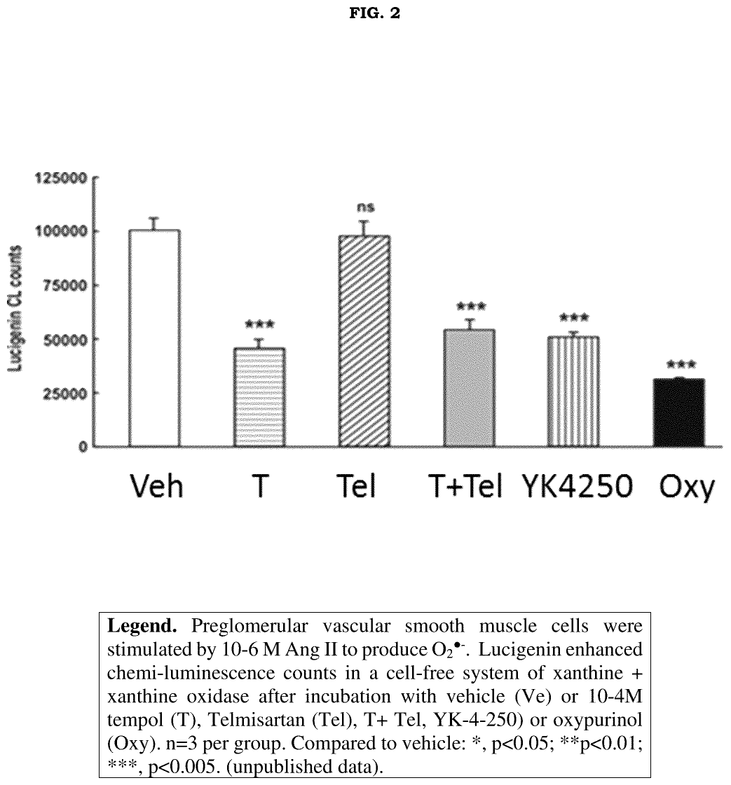

YK-4-250 is a potent antioxidant and inhibits reactive oxygen species in tissues. Preglomerular vascular smooth muscle cells were stimulated by 10⁻⁶ M Ang II to produce O₂⁻. YK-4-250 inhibits O₂₋ generation and is a potent antioxidant. In contrast, Ang II blockers like Telmisartan do not have antioxidant actions (FIG. 2).

ARB mitigates severe acute lung failure induced by SARS-CoV infection in vivo. Ang II AT is the crucial receptor that mediates Ang II-induced vascular permeability and severe acute lung injury.^{1,3} Inhibition of the AT1 indeed attenuated acute severe lung injury in Spike-Fc-treated mice. Inhibition of the AT1R also attenuated pulmonary edema. Taken together, data suggest that SARS-CoV Spike can exaggerate acute lung failure through deregulation of the renin-angiotensin system. Moreover, SARS-CoV Spike-mediated lung failure can be rescued by inhibition of Ang II AT1 receptors. The Ang II AT1 receptor controls acute lung injury severity and pulmonary vascular permeability.

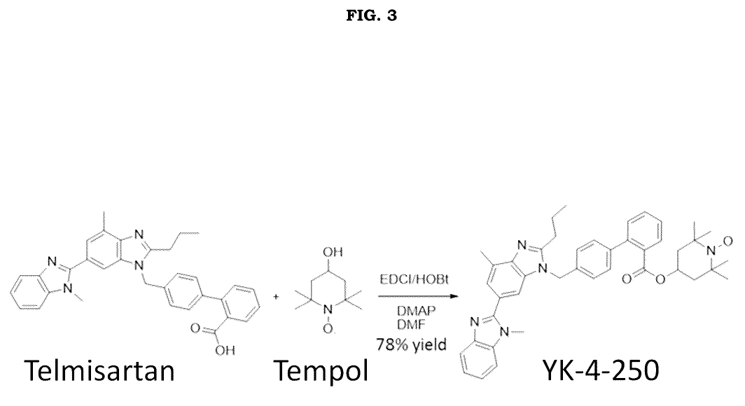

Disclosed herein is the manufacturing feasibility of YK-4-250. Excellent manufacturing feasibility can be achieved with regard to the candidate therapeutic, YK-4-250. In an example embodiment, Tempol was added to Telmisartan in a one-pot synthesis to yield 0.81 grams of YK-4-250 (FIG. 3) as a pink soft solid in 78% yield. The compound molecular weight was confirmed and purity determined by time of flight, high-resolution mass spectrometry. This reaction is scalable to kilogram GMP production.

Examples of the RAS modulators, pharmaceutical compositions, and methods for treating viral infections include at least the following.

RAS modulators may include ARB-antioxidant conjugates, although example embodiments are not limited thereto and may include other types of conjugates as disclosed herein.

For instance, in one type of ARB-antioxidant conjugate, Telmisartan (ARB) may be added through an ester, an ether, an amide, or other bond to Tempol (antioxidant) in an effort to extend the half-life of Tempol (Example is a conjugate such as YK-4-250).

An ARB and an antioxidant may be utilized for inhibiting viral infections including COVID19, coronavirus, and other viruses.

An ARB and an antioxidant may be utilized for inhibiting the reactive oxygen species induced by viral infections.

An ARB and an antioxidant may be utilized for inhibiting cytokine release in viral infections.

An ARB and an antioxidant may be utilized for upregulating ACE2 and/or downregulating AT1.

An ARB and an antioxidant may be utilized for inhibiting the progression of viral diseases.

An ARB and an antioxidant may be utilized for inhibiting acute respiratory distress syndrome (ARDS).

An ARB and an antioxidant may be utilized for inhibiting cardiac dysfunction.

An ARB and an antioxidant may be utilized for inhibiting oral, respiratory, renal, and gastrointestinal injury related to viral infections.

An ARB and an antioxidant may be utilized for preventing the loss or restoration of taste and/or smell.

An ARB, an antioxidant, and a HMG-CoA reductase inhibitor (e.g., Rosuvastatin) may be utilized for inhibiting viral infections including COVID19, coronavirus, and other viruses.

An ARB, an antioxidant, and a HMG-CoA reductase inhibitor may be utilized for inhibiting the reactive oxygen species induced by viral infections.

An ARB, an antioxidant, and a HMG-CoA reductase inhibitor may be utilized for inhibiting cytokine release in viral infections.

An ARB, an antioxidant, and a HMG-CoA reductase inhibitor may be utilized for upregulating ACE2 and/or downregulating AT1.

An ARB, an antioxidant, and a HMG-CoA reductase inhibitor may be utilized for inhibiting the progression of viral diseases.

An ARB, an antioxidant, and a HMG-CoA reductase inhibitor may be utilized for inhibiting acute respiratory distress syndrome (ARDS).

An ARB, an antioxidant, and a HMG-CoA reductase inhibitor may be utilized for inhibiting cardiac dysfunction.

An ARB, an antioxidant, and a HMG-CoA reductase inhibitor may be utilized for inhibiting oral, respiratory, renal, and gastrointestinal injury related to viral infections.

An ARB, an antioxidant, and a HMG-CoA reductase inhibitor may be utilized for preventing the loss or restoration of taste and/or smell.

An ARB and a HMG-CoA reductase inhibitor may be utilized for inhibiting viral infections including COVID19, coronavirus, and other viruses.

An ARB and a HMG-CoA reductase inhibitor may be utilized for inhibiting the reactive oxygen species induced by viral infections.

An ARB and a HMG-CoA reductase inhibitor may be utilized for inhibiting cytokine release in viral infections.

An ARB and a HMG-CoA reductase inhibitor may be utilized for upregulating ACE2 and/or downregulating AT1.

An ARB and a HMG-CoA reductase inhibitor may be utilized for inhibiting the progression of viral diseases.

An ARB and a HMG-CoA reductase inhibitor in combination in a single delivery oral vehicle (e.g., Telmisartan and Rosuvastatin) may be utilized for inhibiting acute respiratory distress syndrome (ARDS).

An ARB and a HMG-CoA reductase inhibitor in combination in a sc, iv, or depot or transdermal administration may be utilized for inhibiting acute respiratory distress syndrome (ARDS).

An ARB and a HMG-CoA reductase inhibitor may be utilized for inhibiting cardiac dysfunction.

An ARB and a HMG-CoA reductase inhibitor in combination in a single delivery oral vehicle may be utilized for inhibiting oral, respiratory, renal, and gastrointestinal injury related to viral infections.

An ARB and a HMG-CoA reductase inhibitor in combination in a sc, iv, or depot or transdermal administration may be utilized for inhibiting oral, respiratory, renal, and gastrointestinal injury related to viral infections.

An ARB and a HMG-CoA reductase inhibitor may be utilized for preventing the loss or restoration of taste and/or smell.

Compositions of matter are also disclosed for the following structures with examples in I, II, III, and IV.

Disclosed below are novel antioxidant-HMG-CoA conjugates (e.g., Tempol-HMG-CoA conjugates) and example I, TP-1-01.

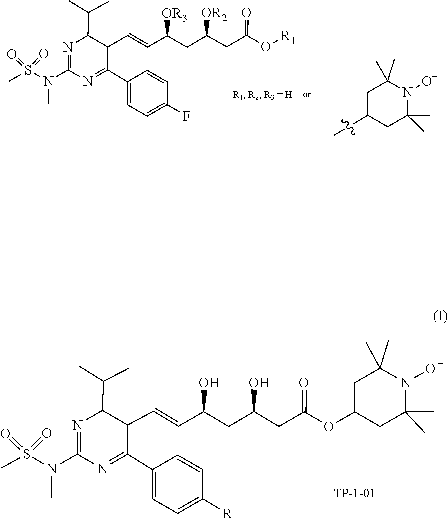

Tempol-HMG-CoA conjugates, as in example I, may be utilized for inhibiting viral infections including COVID19, coronavirus, and other viruses.

Tempol-HMG-CoA conjugates, as in example I, may be utilized for inhibiting the reactive oxygen species induced by viral infections.

Tempol-HMG-CoA conjugates, as in example I, may be utilized for inhibiting cytokine release in viral infections.

Tempol-HMG-CoA conjugates, as in example I, may be utilized for upregulating ACE2 and downregulating AT1.

Tempol-HMG-CoA conjugates, as in example I, may be utilized for inhibiting the progression of viral diseases.

Tempol-HMG-CoA conjugates, as in example I, may be utilized for inhibiting acute respiratory distress syndrome (ARDS).

Tempol-HMG-CoA conjugates, as in example I, may be utilized for inhibiting cardiac dysfunction.

Tempol-HMG-CoA conjugates, as in example I, may be utilized for inhibiting oral, respiratory, renal, and gastrointestinal injury related to viral infections.

Tempol-HMG-CoA conjugates, as in example I, may be utilized for preventing the loss or restoration of taste and/or smell.

Disclosed below are antioxidant-cyclic Ang 1-7 conjugates (e.g., Tempol-cyclic Ang 1-7 conjugates) and example II, TP-2-01.

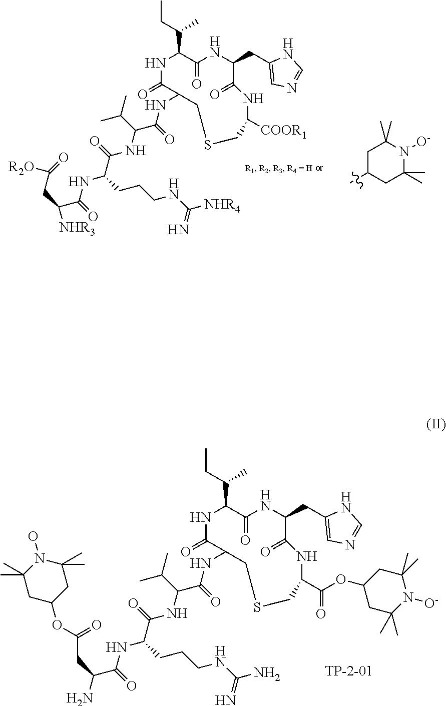

Tempol-cyclic Ang 1-7 conjugates, as in example II, may be utilized for inhibiting viral infections including COVID19, coronavirus, and other viruses.

Tempol-cyclic Ang 1-7 conjugates, as in example II, may be utilized for inhibiting the reactive oxygen species induced by viral infections.

Tempol-cyclic Ang 1-7 conjugates, as in example II, may be utilized for inhibiting cytokine release in viral infections.

Tempol-cyclic Ang 1-7 conjugates, as in example II, may be utilized for upregulating ACE2 and downregulating AT1.

Tempol-cyclic Ang 1-7 conjugates, as in example II, may be utilized for inhibiting the progression of viral diseases.

Tempol-cyclic Ang 1-7 conjugates, as in example II, may be utilized for inhibiting acute respiratory distress syndrome (ARDS).

Tempol-cyclic Ang 1-7 conjugates, as in example II, may be utilized for inhibiting cardiac dysfunction.

Tempol-cyclic Ang 1-7 conjugates, as in example II, may be utilized for inhibiting oral, respiratory, renal, and gastrointestinal injury related to viral infections.

Tempol-cyclic Ang 1-7 conjugates, as in example II, may be utilized for preventing the loss or restoration of taste and/or smell.

Disclosed below are anti-oxidant-Ang 1-7 conjugates (e.g., Tempol-Ang 1-7 conjugates) and example III, TP-03-01.

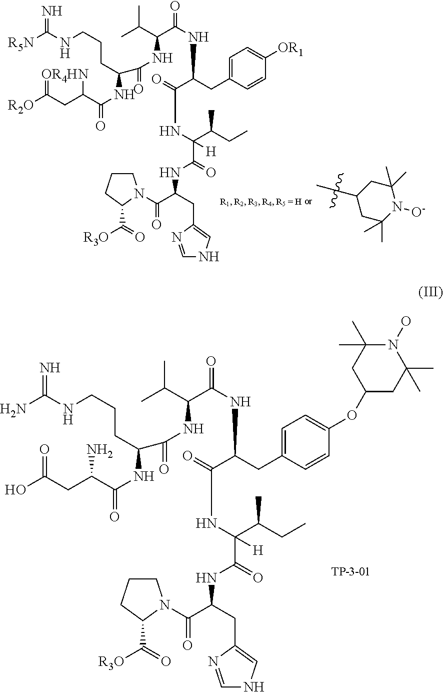

Tempol-Ang 1-7 conjugates, as in example III, may be utilized for inhibiting viral infections including COVID19, coronavirus, and other viruses.

Tempol-Ang 1-7 conjugates, as in example III, may be utilized for inhibiting the reactive oxygen species induced by viral infections.

Tempol-Ang 1-7 conjugates, as in example III, may be utilized for inhibiting cytokine release in viral infections.

Tempol-Ang 1-7 conjugates, as in example III, may be utilized for upregulating ACE2 and downregulating AT1.

Tempol-Ang 1-7 conjugates, as in example III, may be utilized for inhibiting the progression of viral diseases.

Tempol-Ang 1-7 conjugates, as in example III, may be utilized for inhibiting acute respiratory distress syndrome (ARDS).

Tempol-Ang 1-7 conjugates, as in example III, may be utilized for inhibiting cardiac dysfunction.

Tempol-Ang 1-7 conjugates, as in example III, may be utilized for inhibiting oral, respiratory, renal, and gastrointestinal injury related to viral infections.

Tempol-Ang 1-7 conjugates, as in example III, may be utilized for preventing the loss or restoration of taste and/or smell.

Disclosed below are novel antioxidant-Angiotensin converting enzyme (ACE) conjugates (e.g., Tempol-ACE conjugates) and example IV, TP-4-01.

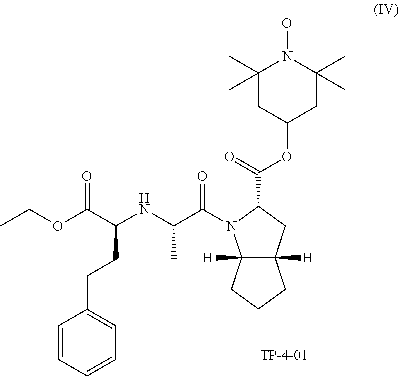 [SMILES: CCOC(=O)[C@H](CCc1ccccc1)N[C@@H](C)C(=O)N1[C@H](C(=O)OC2CC(C)(C)N(O)C(C)(C)C2)C[C@@H]2CCC[C@@H]21]

ACE inhibitor or Tempol-ACE conjugates, as in example IV, may be utilized for inhibiting viral infections including COVID19, coronavirus, and other viruses.

ACE inhibitor or Tempol-ACE conjugates, as in example IV, may be utilized for inhibiting the reactive oxygen species induced by viral infections.

ACE inhibitor or Tempol-ACE conjugates, as in example IV, may be utilized for inhibiting cytokine release in viral infections.

ACE inhibitor or Tempol-ACE conjugates, as in example IV, may be utilized for upregulating ACE2 and/or downregulating AT1.

ACE inhibitor or Tempol-ACE conjugates, as in example IV, may be utilized for inhibiting the progression of viral diseases.

ACE inhibitor or Tempol-ACE conjugates, as in example IV, may be utilized for inhibiting acute respiratory distress syndrome (ARDS).

ACE inhibitor or Tempol-ACE conjugates, as in example IV, may be utilized for inhibiting cardiac dysfunction.

ACE inhibitor or Tempol-ACE conjugates, as in example IV, may be utilized for inhibiting oral, respiratory, renal, and gastrointestinal injury related to viral infections.

ACE inhibitor or Tempol-ACE conjugates, as in example IV, may be utilized for preventing the loss or restoration of taste and/or smell.

Disclosed below are novel antioxidant-3,3′-diindolylmethane (DIM) conjugates (e.g., Tempol-DIM conjugates) and example V, TP-5-01.

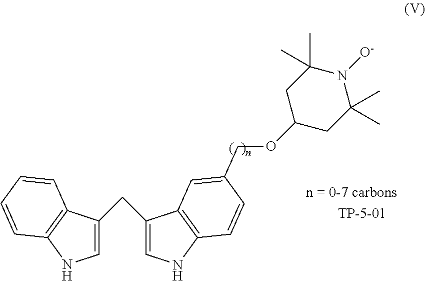

DIM or Tempol-DIM conjugates, as in example V, may be utilized for inhibiting viral infections including COVID19, coronavirus, and other viruses.

DIM or Tempol-DIM conjugates, as in example V, may be utilized for inhibiting the reactive oxygen species induced by viral infections.

DIM or Tempol-DIM conjugates, as in example V, may be utilized for inhibiting cytokine release in viral infections.

DIM or Tempol-DIM conjugates, as in example V, may be utilized for upregulating ACE2 and/or downregulating AT1.

DIM or Tempol-DIM conjugates, as in example V, may be utilized for inhibiting the progression of viral diseases.

DIM or Tempol-DIM conjugates, as in example V, may be utilized for inhibiting acute respiratory distress syndrome (ARDS).

DIM or Tempol-DIM conjugates, as in example V, may be utilized for inhibiting cardiac dysfunction.

DIM or Tempol-DIM conjugates, as in example V, may be utilized for inhibiting oral, respiratory, renal, and gastrointestinal injury related to viral infections.

DIM or Tempol-DIM conjugates, as in example V, may be utilized for preventing the loss or restoration of taste and/or smell.

Disclosed below are novel antioxidant-indole-3-carbinol (I3C) conjugates (e.g., Tempol-I3C conjugates) and example VI, TP-6-01.

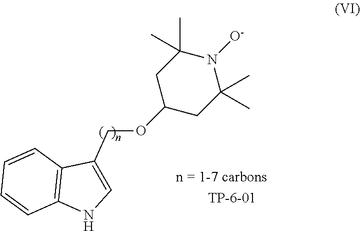

I3C or Tempol-I3C conjugates, as in example VI, may be utilized for inhibiting viral infections including COVID19, coronavirus, and other viruses.

I3C or Tempol-I3C conjugates, as in example VI, may be utilized for inhibiting the reactive oxygen species induced by viral infections.

I3C or Tempol-I3C conjugates, as in example VI, may be utilized for inhibiting cytokine release in viral infections.

I3C or Tempol-I3C conjugates, as in example VI, may be utilized for upregulating ACE2 and/or downregulating AT1.

I3C or Tempol-I3C conjugates, as in example VI, may be utilized for inhibiting the progression of viral diseases.

I3C or Tempol-I3C conjugates, as in example VI, may be utilized for inhibiting acute respiratory distress syndrome (ARDS).

I3C or Tempol-I3C conjugates, as in example VI, may be utilized for inhibiting cardiac dysfunction.

I3C or Tempol-I3C conjugates, as in example VI, may be utilized for inhibiting oral, respiratory, renal, and gastrointestinal injury related to viral infections.

I3C or Tempol-I3C conjugates, as in example VI, may be utilized for preventing the loss or restoration of taste and/or smell.

I3C is a precursor to DIM. As a result, when administered (e.g., orally) to a patient/subject in need thereof, I3C will be converted to DIM. Similarly, Tempol-I3C conjugates will be converted to Tempol-DIM conjugates when administered to such a patient/subject.

Disclosed below are novel antioxidant-pirfenidone (PFD) conjugates (e.g., Tempol-PFD) and example VII, TP-7-01.

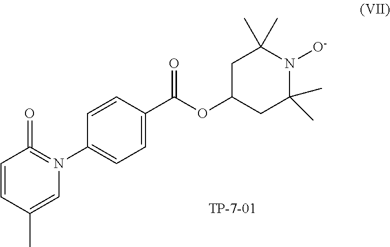

Tempol-PFD conjugates, as in example VII, may be utilized for inhibiting viral infections including COVID19, coronavirus, and other viruses.

Tempol-PFD conjugates, as in example VII, may be utilized for inhibiting the reactive oxygen species induced by viral infections.

Tempol-PFD conjugates, as in example VII, may be utilized for inhibiting cytokine release in viral infections.

Tempol-PFD conjugates, as in example VII, may be utilized for upregulating ACE2 and/or downregulating AT1.

Tempol-PFD conjugates, as in example VII, may be utilized for inhibiting the progression of viral diseases.

Tempol-PFD conjugates, as in example VII, may be utilized for inhibiting acute respiratory distress syndrome (ARDS).

Tempol-PFD conjugates, as in example VII, may be utilized for inhibiting cardiac dysfunction.

Tempol-PFD conjugates, as in example VII, may be utilized for inhibiting oral, respiratory, renal, and gastrointestinal injury related to viral infections.

Tempol-PFD conjugates, as in example VII, may be utilized for preventing the loss or restoration of taste and/or smell.

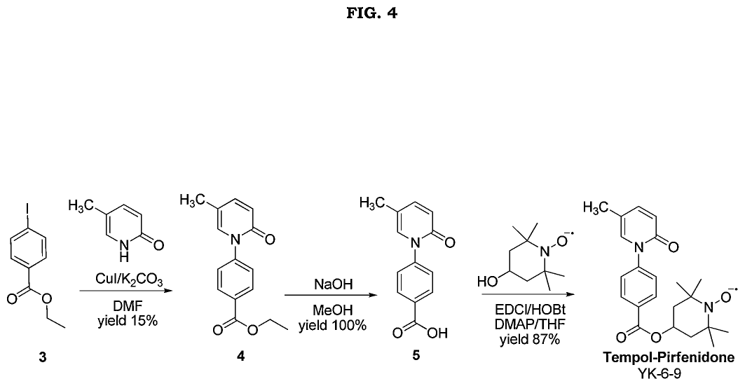

FIG. 4 illustrates the synthesis of an antioxidant-TGF-β inhibitor (e.g., Tempol-TGF-β inhibitor). Referring to FIG. 4, the synthesis of Tempol-Pirfenidone (YK-6-9) may start with the addition of 5-methylpyridin-2(1H)-one to ethyl 4-iodobenzoate 3 to generate compound 4. Saponification of 4 results in the acid 5 in quantitative yield. Esterification may be completed by the addition of Tempol to generate YK-6-9 in 87% yield.

Selected herein for purposes of discussion are validated molecular targets that, upon inhibition by specific FDA approved drugs, up-regulate the cytoprotective ACE2 (FIG. 1). These include inhibitors of AT1 (ARBs),¹⁴ HMG-CoA reductase (statins),¹⁵ TGF-β inhibitor (Pirfenidone)¹⁶, and ACEI.¹⁷

The above structures, with examples in I, II, III, IV, V, VI, and VII, may be additionally utilized in radiation mitigation, radiation protection, reduction of oxidative stress, reduction in blood pressure, prevention or reduction in fibrosis (e.g. radiation-induced fibrosis, chemical-induced fibrosis, viral-induced fibrosis, cancer-induced fibrosis, idiopathic fibrosis), prevention or mitigation of chronic kidney disease, prevention or mitigation of inflammatory bowel disease, enhancement of immunotherapy, organ transplant, cancer treatment, and Alzheimer's disease.

The above structures, with examples in I, II, III, IV, V, VI, and VII, may be further utilized in the treatment of neurodegenerative diseases, neovascular diseases, and inflammatory diseases of the eye, including glaucoma, age-related macular degeneration, diabetic retinopathy, and retinopathy of prematurity.

In view of the teachings herein, it should be understood that the various examples may be used individually or in combination for medical treatment. For instance, there are at least the following examples, as summarized in the tables below, although example embodiments are not limited thereto. In the tables below, it should ¹⁴ Igase, M., Kohara, K., Nagai, T. et al. Increased Expression of Angiotensin Converting Enzyme 2 in Conjunction with Reduction of Neointima by Angiotensin II Type 1 Receptor Blockade. Hypertens Res 31, 553-559 (2008). https://doi.org/10.1291/hypres.31.553.¹⁵ Li Y H, Wang Q X, Zhou J W, Chu X M, Man Y L, Liu P, Ren B B, Sun T R, An Y. Effects of rosuvastatin on expression of angiotensin-converting enzyme 2 after vascular balloon injury in rats. J Geriatr Cardiol. 2013 Jun.;10(2):151-8. doi: 10.3969/j.issn.1671-5411.2013.02.009. PMID: 23888175; PMCID: PMC3708055.¹⁶ Cho M E, Kopp J B. Pirfenidone: an anti-fibrotic therapy for progressive kidney disease. Expert Opin Investig Drugs. 2010; 19(2):275-283. doi:10.1517/13543780903501539.¹⁷ Huang M L, Li X, Meng Y, et al. Upregulation of angiotensin-converting enzyme (ACE) 2 in hepatic fibrosis by ACE inhibitors. Clin Exp Pharmacol Physiol. 2010; 37(1):e1-e6. doi:10.1111/j.1440-1681.2009.05302.x.

be understood that a “+” indicates a link/bond, while an “&” indicates that the compounds are present together (e.g., for co-administration) but not linked via a chemical bond. Additionally, it should be understood that, in all instances indicated by DIM, its precursor (I3C) may be included with or interchanged with DIM.

Use as Antivirals

| | Angiotensin (1-7) | | ACE Inhibitors | | |
|---|---|---|---|---|---|
| ARB | (Ang 1-7) | HMG-CoA | (ACE) | DIM | PFD |
| ARB + | Ang 1-7 + T | HMG-CoA + T | ACE + T | DIM + T | PFD + T |
| Tempol (T) | Cyclic Ang 1-7 | | | | |
| | Cyclic Ang 1-7 + T | | | | |

Additional uses (radiation mitigation, radiation protection, reduction of oxidative stress, reduction in blood pressure, prevention or reduction in fibrosis, prevention or mitigation of chronic kidney disease, prevention or mitigation of inflammatory bowel disease, enhancement of immunotherapy, organ transplant, cancer treatment, Alzheimer's disease, and treatment of eye-related neurodegenerative, neovascular and inflammatory diseases.)

| | | | | | |
|---|---|---|---|---|---|
| HMG-CoA + T | Ang 1-7 + T | Cyclic Ang 1-7 + T | ACE + T | DIM + T | PFD + T |

Composition (Co-Administered)

| | | | |
|---|---|---|---|
| ARB & HMG-CoA | ARB & Ang 1-7 | ACE & HMG-CoA | ACE & Ang 1-7 |
| ARB & HMG-CoA + T | ARB & Ang 1-7 + T | ACE & HMG-CoA + T | ACE & Ang 1-7 + T |
| ARB + T & HMG-CoA | ARB + T & Ang 1-7 | ACE + T & HMG-CoA | ACE + T & Ang 1-7 |
| ARB + T & HMG-CoA + T | ARB + T & Ang 1-7 + T | ACE + T & HMG-CoA + T | ACE + T & Ang 1-7 + T |
| ARB & DIM | ARB & Cyclic Ang 1-7 | ACE & DIM | ACE & Cyclic Ang 1-7 |
| ARB & DIM + T | ARB & Cyclic Ang 1-7 + T | ACE & DIM + T | ACE & Cyclic Ang 1-7 + T |
| ARB + T & DIM | ARB + T & Cyclic Ang 1-7 | ACE + T & DIM | ACE + T & Cyclic Ang 1-7 |
| ARB + T & DIM + T | ARB + T & Cyclic Ang 1-7 + T | ACE + T & DIM + T | ACE + T & Cyclic Ang 1-7 + T |
| ARB & PFD | DIM & Ang 1-7 | ACE & PFD | PFD & Ang 1-7 |
| ARB & PFD + T | DIM & Ang 1-7 + T | ACE & PFD + T | PFD & Ang 1-7 + T |
| ARB + T & PFD | DIM + T & Ang 1-7 | ACE + T & PFD | PFD + T & Ang 1-7 |
| ARB + T & PFD + T | DIM + T & Ang 1-7 + T | ACE + T & PFD + T | PFD + T & Ang 1-7 + T |
| HMG & DIM | DIM & Cyclic Ang 1-7 | HMG & PFD | PFD & Cyclic Ang 1-7 |
| HMG & DIM + T | DIM & Cyclic Ang 1-7 + T | HMG & PFD + T | PFD & Cyclic Ang 1-7 + T |
| HMG + T & DIM | DIM + T & Cyclic Ang 1-7 | HMG + T & PFD | PFD + T & Cyclic Ang 1-7 |
| HMG + T & DIM + T | DIM + T & Cyclic Ang 1-7 + T | HMG + T & PFD + T | PFD + T & Cyclic Ang 1-7 + T |

The overwhelming and catastrophic damage to global health and the economy by COVID-19 requires novel approaches to develop safe and effective therapies to treat this multi-organ infection. New drugs have been identified herein that boost the infected patient's ability to reverse the destructive effects of COVID-19 viral entry into the lung, GI, and cardiovascular system. This strategy is the first potential treatment that does not rely on blocking the virus from entering the tissue or attempting to kill the virus. Instead, the strategy focuses on preventing the surge of detrimental downstream cellular and organ effects mediated by the virus. This strategy is in sharp contrast to traditional approaches aimed at attacking the virus.

Currently, patients that are infected with COVID-19 unfortunately seek medical care at a relatively late stage, often when they are unable to breathe. These patients need oxygen due to severe lung injury and pneumonia. A sign of major and sometimes dismal outcome occurs when the patients have to be placed on a ventilator. As the damage continues to affect all major organ systems including the heart, many patients will ultimately die. In fact, an alarming number of patients die before reaching the hospital. The therapeutic approach taught herein which blocks these major complications will have a significant impact on disease severity, improved survival and allow patients to recover. An added advantage to the treatment herein is the prevention of the long-term complications that are associated with COVID-19 patients that currently survive the infection.

As disclosed herein according to an example embodiment, an angiotensin receptor blocker (ARB), e.g., Telmisartan, may be the central inhibitor of the Ang II mediated cell toxicity, restoring the cells ability to re-activate its own defense. A first strategy combines the ARB with a highly potent antioxidant in one molecule to also stop the damaging effects of ROS and the surge in cytokine release.

A second strategy combines the ARB with a highly potent anti-inflammatory drug HMG-CoA reductase inhibitor (e.g., Rosuvastatin) that acts by upregulating ACE2 and downregulating AT1. Additional combinations include, but are not limited to, 3,3′-diindolylmethane (DIM), indole-3-carbinol (I3C), and/or pirfenidone (PFD).

The documents cited in the footnotes herein, a list of which is provided below, are incorporated herein by reference in their entirety.
Chang X, Firestone G L, Bjeldanes L F. Inhibition of growth factor-induced Ras signaling in vascular endothelial cells and angiogenesis by 3,3′-diindolylmethane. Carcinogenesis. 2006 Mar;27(3):541-50. doi: 10.1093/carcin/bgi230. Epub 2005 Sep. 30. PMID: 16199440.
Shi, K., Wang, F., Xia, J., Zuo, B., Wang, Z., Cao, X. (2019). Pirfenidone inhibits epidural scar fibroblast proliferation and differentiation by regulating TGF-β1-induced. Smad-dependent and -independent pathways. American journal of translational research, 11(3), 1593-4604.
Imai Y, Kuba K, Rao S, et al. Angiotensin-converting enzyme 2 protects from severe acute lung failure. Nature. 2005; 436(7047):112-116. doi:10.1038/nature03712.
Lei Fang, George Karakiulakis, Michael Roth. Are patients with hypertension and diabetes mellitus at increased risk for COVID-19 infection? Lancet. Mar. 11, 2020DOI:https://doi.org/10.1016/S2213-2600(20)30116-8.
Kuba, K., Imai, Y., Rao, S. et al. A crucial role of angiotensin converting enzyme 2 (ACE2) in SARS coronavirus-induced lung injury. Nat Med 11, 875-879 (2005). https://doi.org/10.1038/nm1267.
Wang J, Zhao S, Liu M, et al. ACE2 expression by colonic epithelial cells is associated with viral infection, immunity and energy metabolism, 2020.
Marshall R P. The pulmonary renin-angiotensin system. Curr Pharm Des 2003; 9:715-22.
Benigni A, Cassis P, Remuzzi G. Angiotensin II revisited: new roles in inflammation, immunology and aging. EMBO Mol Med 2010; 2:247-57.
Guo T, Fan Y, Chen M, et al. Cardiovascular Implications of Fatal Outcomes of Patients With Coronavirus Disease 2019 (COVID-19). JAMA Cardiol. Mar. 27, 2020. doi:10.1001/jamacardio.2020.1017.
Brown M L, Kong, Y., Wilcox, C. S. Treatment for oxidative stress and/or hypertension. In: Patent US, ed. USPTO. USA: Georgetown University (Washington, DC), 2016.
David Olagnier, et al Cellular Oxidative Stress Response Controls the Antiviral and Apoptotic Programs in Dengue Virus-Infected Dendritic Cells. PLoS Pathog. 2014 December; 10(12): e1004566.
Aziz Guellich, Thibaud Damy, Marc Conti, Victor Claes, Jane-Lise Samuel, Thierry Pineau, Yves Lecarpentier, Catherine Coirault. Tempol Prevents Cardiac Oxidative Damage and Left Ventricular Dysfunction in the PPAR-α KO Mouse. Am J Physiol Heart Circ Physiol, 304 (11), H1505-12 2013.
Kuppusamy P, Wang P, Shankar R A, et al. In vivo topical EPR spectroscopy and imaging of nitroxide free radicals and polynitroxyl-albumin. Magn Reson Med 1998; 40:806-11.
Igase, M., Kohara, K., Nagai, T. et al. Increased Expression of Angiotensin Converting Enzyme 2 in Conjunction with Reduction of Neointima by Angiotensin II Type 1 Receptor Blockade. Hypertens Res 31, 553-559 (2008). https://doi.org/10.1291/hypres.31.553.
Li Y H, Wang Q X, Zhou J W, Chu X M, Man Y L, Liu P, Ren B B, Sun T R, An Y. Effects of rosuvastatin on expression of angiotensin-converting enzyme 2 after vascular balloon injury in rats. J Geriatr Cardiol. 2013 June;10(2):151-8. doi: 10.3969/j.issn.1671-5411.2013.02.009. PMID: 23888175; PMCID: PMC3708055.
Cho M E, Kopp J B. Pirfenidone: an anti-fibrotic therapy for progressive kidney disease. Expert Opin Investig Drugs. 2010; 19(2):275-283. doi:10.1517/13543780903501539.
Huang M L, Li X, Meng Y, et al. Upregulation of angiotensin-converting enzyme (ACE) 2 in hepatic fibrosis by ACE inhibitors. Clin Exp Pharmacol Physiol. 2010; 37(1):e1-e6. doi:10.1111/j.1440-1681.2009.05302.x.

While a number of example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

1-42. (canceled)

43. A pharmaceutical composition for treating subject in need thereof, comprising:
a renin-angiotensin system (RAS) modulator including pirfenidone (PFD) linked to a first antioxidant, the first antioxidant including tempol; and
a pharmaceutically-acceptable carrier.

44. The pharmaceutical composition of claim 43, wherein the renin-angiotensin system modulator further includes at least one of an angiotensin receptor blocker (ARB), angiotensin (1-7), a HMG-CoA reductase inhibitor, or an angiotensin-converting-enzyme (ACE) inhibitor.

45. The pharmaceutical composition of claim 44, wherein the angiotensin receptor blocker includes telmisartan.

46. The pharmaceutical composition of claim 44, wherein the angiotensin (1-7) includes cyclic Ang 1-7.

47. The pharmaceutical composition of claim 44, wherein the HMG-CoA reductase inhibitor includes rosuvastatin.

48. The pharmaceutical composition of claim 44, wherein at least one of the angiotensin receptor blocker, the angiotensin (1-7), the HMG-CoA reductase inhibitor, or the angiotensin-converting-enzyme inhibitor is linked to a second antioxidant, the second antioxidant including tempol.

49. The pharmaceutical composition of claim 48, wherein the renin-angiotensin system modulator includes the pirfenidone linked to the first antioxidant and the angiotensin receptor blocker linked to the second antioxidant.

50. The pharmaceutical composition of claim 48, wherein the renin-angiotensin system modulator includes the pirfenidone linked to the first antioxidant and the angiotensin (1-7) linked to the second antioxidant.

51. The pharmaceutical composition of claim 48, wherein the renin-angiotensin system modulator includes the pirfenidone linked to the first antioxidant and the HMG-CoA reductase inhibitor linked to the second antioxidant.

52. The pharmaceutical composition of claim 48, wherein the renin-angiotensin system modulator includes the pirfenidone linked to the first antioxidant and the angiotensin-converting-enzyme inhibitor linked to the second antioxidant.

53. A method of inhibiting viral infections, comprising:
administering the pharmaceutical composition of claim 43 to the subject in need thereof.

54. A method of inhibiting reactive oxygen species or reducing oxidative stress, comprising:
administering the pharmaceutical composition of claim 43 to the subject in need thereof.

55. A method of inhibiting cytokine release, comprising:
administering the pharmaceutical composition of claim 43 to the subject in need thereof.

56. A method of mitigating or protecting against radiation, comprising:
administering the pharmaceutical composition of claim 43 to the subject in need thereof.

57. A method of preventing or reducing fibrosis, comprising:
administering the pharmaceutical composition of claim 43 to the subject in need thereof.

58. A method of treating diseases of the eye, comprising:
administering the pharmaceutical composition of claim 43 to the subject in need thereof.
